(19) United States
(12) Patent Application Publication
(10) Pub. No.: US 2025/0288384 A1
(43) Pub. Date: Sep. 18, 2025

(54) PERFORATED INSTRUMENT HOLDER FOR MEDICAL INSTRUMENTS, IN PARTICULAR SURGICAL INSTRUMENTS

(71) Applicant: Aesculap AG, Tuttlingen (DE)

(72) Inventors: Daniel Weisser, Villingen-Schwenningen (DE); Eva Streit, Bodman-Ludwigshafen (DE); Timo Knittel, Wurmlingen (DE); Hermann Deutscher, Neuhausen ob Eck (DE)

(21) Appl. No.: 19/223,901

(22) Filed: May 30, 2025

Related U.S. Application Data
(63) Continuation of application No. PCT/EP2024/053052, filed on Feb. 7, 2024.

(30) Foreign Application Priority Data
Feb. 14, 2023 (DE) 10 2023 103 493.6

(51) Int. Cl. A61B 50/20

(52) U.S. Cl. CPC A61B 50/20

(57) ABSTRACT

A holder system for medical instruments includes a support structure having openings, a top side, and a device. The device is positionable at various locations on the top side for holding instruments. The device includes a base part and an instrument bearing part releasably fastenable to the base part. The base part has a main limb that is placeable against the top side and two side limbs adjoining the main limb. Each side limb has at least one flexible tongue that is deflectable. The base part is securable on the support structure in such a way that the flexible tongue engages a bottom side of the support structure through an opening. By exerting a compressive force on the base part, a flexible tongue can be deflected and penetrate into an opening to latch with an edge region that delimits the opening.

### CROSS-REFERENCE TO RELATED APPLICATIONS

This application is a continuation under 35 U.S.C. § 365 of International Application No. PCT/EP2024/053052, filed on Feb. 7, 2024, which claims priority to German Application No. 10 2023 103 493.6, filed on Feb. 14, 2023. The contents of International Application No. PCT/EP2024/053052 and German Application No. 10 2023 103 493.6 are incorporated by reference herein in their entireties.

### FIELD

The present disclosure relates to a perforated instrument holder for medical instruments, in particular surgical instruments and/or implants for use in supplying sterile goods in the medical field, in particular operating theaters, comprising a perforated holder support structure having a plurality of perforated holder openings, having a top side, which faces the instruments, and a bottom side, which faces away from the instruments, of the perforated holder support structure, having at least one device that can be positioned from the top side of the perforated holder support structure at various selectable locations on the top side for storing and preferably for holding one or more instruments in an intended arrangement on the perforated instrument holder, wherein the device comprises a base part and an instrument bearing part that can be releasably fastened thereto, wherein the base part can be releasably secured to the perforated holder support structure.

### BACKGROUND

Such perforated instrument holders, which are often designed as perforated instrument baskets or perforated holder inserts for sterile goods containers, play an important role in the so-called sterile goods cycle of medical and in particular surgical instruments since they receive the instruments to be sterilized during sterilization and also afterward during transport to the sterile goods storage facility and from there to the place of use, in particular an operating theater. For this purpose, so-called organization systems for perforated instrument holders of the type mentioned above have already been proposed for keeping the instruments in a predetermined position and arrangement on the perforated instrument holders until they are removed by medical personnel immediately before they are used. Previously known devices for storing one or more instruments on the perforated instrument holder can either be fastened to the perforated instrument holder or its perforated holder support structure by means of a screw connection, which is considered to be complex, or are inserted through the perforated holder support structure from the bottom side of the perforated holder support structure through perforated holder openings or other specially designed openings such that they protrude upward on the top side of the perforated instrument holder in order to be able to store instruments there. Both imply that the retaining device is to some extent assembled on both sides of the perforated instrument holder. The retaining device or devices can therefore only be configured on the perforated holder support structure when commencing with loading instruments, since the perforated instrument holder must be tilted and turned for this purpose. Mounting elements arranged on the bottom side of the perforated instrument holder have also proven to be disruptive; in particular, they can lead to abrasion, damage to the support surface or, if not distributed uniformly, to the perforated instrument holder wobbling on the support surface.

US 2020/0030049A1 discloses a carrier for items to be sterilized in which posts with hook portions are clipped into slots on the carrier. Partial elements can then be inserted into the post. DE 697 32 986 T2 discloses an instrument holder in which a main body made of silicone and a stainless steel support frame having ribs and locking prongs is provided.

### SUMMARY

Proceeding therefrom, the object addressed by the present disclosure is that of making a perforated instrument holder of the type mentioned at the outset more user-friendly with regard to the releasable arrangement of the device for storing one or more instruments, the device being releasably fastened to the perforated holder support structure without the use of tools.

This object is achieved according to the present disclosure in a perforated instrument holder of the aforementioned type in that the base part has a main limb that can be placed against the top side of the perforated holder support structure and two opposite side limbs laterally adjoining said main limb, in that, proceeding from each of the side limbs of the base part, at least one flexible tongue and preferably at least two flexible tongues is/are formed on one or both sides, which extend from the side limbs of the base part in the direction of the perforated holder support structure and can be resiliently deflected relative to the side limbs such that the base part can be positioned with its main limb on the top side of the perforated holder support structure in such a way that the at least one flexible tongue engages behind a perforated holder opening, and in that only by exerting a compressive force on the base part in the direction of the top side of the perforated holder support structure is the opposite flexible tongue then deflected and automatically penetrates into another perforated holder opening where it latches with an edge region that delimits the perforated holder opening such that the base part can be fastened to the perforated holder support structure without the use of tools and in a captive manner for the intended use.

It is therefore proposed according to the present disclosure to design the base part of the retaining device to have flexible tongues extending downward, in particular obliquely downward, in the direction of the perforated holder support structure, by means of which tongues the base part can be hooked into one or more perforated holder openings from the top side of the perforated holder support structure by means of the flexible tongue(s) on one side of the base part, and then, merely by pressing on the base part in the direction of the top side of the perforated holder support structure, the flexible tongues provided on the opposite side limb of the base part are deflected and also brought into a position in which it engages behind the perforated holder support structure. In this way, it is conceivable to arrange and assemble each base part on the perforated instrument holder without the use of tools. Following this, the instrument bearing part can then be releasably arranged on the base part, for example by being pushed or placed onto the base part in the direction of the plane of the perforated holder support structure or by being force-fittingly or form-fittingly attached to the base part in another manner. However, it is also conceivable that the base part together with the instrument bearing part already arranged on the base part is releasably secured to the perforated instrument holder in the manner described above.

With regard to economic manufacturability and stability of the device, it has proven to be advantageous if the base part is a bent sheet metal part and the flexible tongues are notched out of the bent sheet metal part. In such a case, the base part including its flexible tongues can be formed from a flat metal material portion by punching and bending processes. A bending fold between the main limb and the two laterally adjacent side limbs gives the base part high torsional stability, even with thin wall thicknesses of the base part of in particular 0.2 to 1.0 mm, in particular 0.25 to 0.8 mm and in particular 0.3 to 0.5 mm. Furthermore, a further fold, preferably an inward fold, can be provided at the top free end of the side limbs, which further increases torsional rigidity and can also limit a sliding seat receptacle for the instrument bearing part.

Furthermore, it has proven to be advantageous if the flexible tongues are notched out of the side limb and the main limb. In such a case, the flexible tongue can be sufficiently long that it can engage in a perforated holder opening of the perforated instrument holder without additional retaining extensions and rear-engagement elements having to be separately provided or attached to the free end of the flexible tongue.

In order to reliably secure the device or its base part to the perforated instrument holder, it has proven to be advantageous for a free end of preferably each of the flexible tongues to be designed or shaped to form a rear-engaging region or a rear-engaging bead. Thus, each flexible tongue is able to reach a form-fitting rear-engagement position with an edge region of the perforated holder support structure that delimits the corresponding perforated holder opening and to also reliably remain therein under the effect of a spring-back force of the flexible tongue.

It has also proven to be advantageous if a free end of preferably each of the flexible tongues is angled by more than 90°, preferably by at least 100°, preferably by at least 110° to the longitudinal extension of the flexible tongue and thus engages behind an edge region delimiting a perforated holder opening.

In order for the operator to secure the base part to the perforated holder support structure in a simple, reliable and convenient manner, it has proven to be advantageous if a free end of preferably each of the flexible tongues forms a run-on slope of the corresponding flexible tongue, which, when the base part is pressed in the direction of the top side of the perforated holder support structure, runs against an edge region delimiting a perforated holder opening and is deflected in order to latch with the edge region into a rear-engagement position.

As already mentioned at the outset, it has proven expedient if the base part forms a latching receptacle or a sliding seat receptacle for the instrument bearing part, wherein the sliding seat receptacle is laterally delimited by the two side limbs of the base part and at the bottom by the main limb. Furthermore, one or preferably both side limbs can have an inward overhang at their top edge region, in particular by the side limb being folded, and thereby delimit the sliding seat receptacle at the top.

According to one embodiment, the instrument bearing part, which can be releasably fastened to the base part, is a polymeric plastics part, which preferably comprises or consists of silicone. Although the instrument bearing part is dimensionally stable, it expediently has flexibly deflectable retaining regions for the instruments. It is expediently individually adapted to instruments to be stored and for this purpose comprises retaining structures, in particular retaining arms and, if necessary, recesses and centering bevels, for placing and, in particular, clipping in instruments.

In principle, the perforated instrument holder can have any perforated surface structure as long as the perforated holder openings are arranged periodically and therefore the arrangement and design, in particular the distance between the retaining tongues, can be designed in accordance with the distance between the perforated holder openings account. With regard to the production of a perforated surface structure that is largely open for sterilization purposes, it has proven to be advantageous if the perforated instrument holder has a perforated holder support structure having in particular rectangular and preferably square perforated holder openings that are delimited by connecting pieces that are narrower than the side length of the rectangular shape or than the largest dimension of the perforated holder openings. In particular, the width of the connecting pieces is not more than 30%, in particular not more than 20%, of the largest dimension of the perforated holder openings, whether they are rectangular or not.

It may also prove to be advantageous for the perforated instrument holder to be rectangular when
the top side is viewed from above, with a corresponding longitudinal direction and transverse direction and for the connecting pieces delimiting the perforated holder openings to also extend at an inclination of 45° to the longitudinal direction or transverse direction. In such a case, the perforated holder openings can also extend at an inclination of 45° to the longitudinal direction or transverse direction of the perforated instrument holder. However, if the base parts are then aligned in the longitudinal direction or transverse direction, to some extent the spring-back force of the flexible tongues leads to self-centering of the relevant flexible tongue in a corner region of a relevant perforated holder opening.

Furthermore, it may prove advantageous if the perforated instrument holder comprises a 3-dimensionally structured, in particular corrugated, perforated holder support structure. In such a case, the base part can be arranged and fastened in the region of high points or peaks of the perforated holder support structure, wherein it does not have to rest on top of these high points or peaks of the perforated holder support structure perfectly, but it is conceivable that the perforated holder support structure engages in recesses in the main limb of the base part by means of its high points or peaks.

In a development of this concept, it has proven to be advantageous if recesses are formed in the base part, in which the perforated holder support structure can engage by means of raised portions such that the position of the base part on the perforated holder support structure can be further stabilized.

In the case of a 3-dimensionally structured, in particular corrugated, perforated holder support structure, the flexible tongues engaging behind the perforated holder openings or their rear-engaging ends are also arranged on the bottom side, but approximately in the region of a raised portion of the perforated holder support structure and thus at a distance from a support surface of the perforated instrument holder. This safely prevents any wear-inducing strain on the support surface caused by the free ends of the flexible tongues.

In a preferred embodiment, the perforated instrument holder including its perforated holder support structure can be formed from a flat metal material, in particular from a metal sheet, and can be manufactured in particular by cutting, punching and bending processes, in particular using laser cutting technology. In this way, the material thickness of the perforated holder support structure can be kept constant over its extension, in particular over the entire bottom region of a perforated basket. In principle, however, woven perforated holder support structures are also conceivable and sometimes advantageous.

With regard to the above-mentioned self-centering of the flexible tongues in corner regions of the perforated holder openings, it has proven to be advantageous if a distance between two adjacent flexible tongues of a side limb corresponds to a distance between two intersection points of the connecting pieces delimiting the perforated holder openings or is twice or an integer multiple thereof, and therefore the flexible tongues can be automatically positioned in respective corner regions of the perforated holder openings.

Furthermore, the present disclosure provides for a release instrument adapted to the base part and its flexible tongues as a component of the perforated instrument holder or as an accessory as such, which can be placed from the inside via openings in the base part against one or more flexible tongues in the region of a side limb and as a result these flexible tongues can be deflected from their rear-engagement position such that the base part can be released from a position in which it engages behind the perforated instrument holder.

As already suggested at the outset, the perforated instrument holder can be used as a perforated instrument basket or as an insert for a perforated instrument basket or instrument container.

As part of an organization system for a perforated instrument holder of the type according to the present disclosure in question here, the present disclosure provides for a device as such for storing and preferably for holding one or more medical instruments, in particular surgical instruments, in an intended arrangement on a perforated instrument holder, wherein the device comprises a base part and an instrument bearing part that can be fastened thereto, wherein the base part can be releasably secured to the perforated instrument holder, the device being characterized in that the base part has a main limb that can be placed against the top side and two opposite side limbs laterally adjoining said main limb, and in that, proceeding from each of the side limbs of the base part, at least one, and preferably two, flexible tongues is/are formed on one or both sides, which extend from the side limbs of the base part in the direction of the perforated holder support structure and can be resiliently deflected relative to the side limbs such that the base part can be positioned with its main limb on the top side of the perforated holder support structure in such a way that the at least one flexible tongue engages behind a perforated holder opening, and in that only by exerting a compressive force on the base part in the direction of the top side of the perforated holder support structure is the opposite flexible tongue then deflected and automatically penetrates into another perforated holder opening where it latches with an edge region that delimits the perforated holder opening such that the base part can be fastened to the perforated holder support structure without the need for tools and in a captive manner for the intended use.

Furthermore, the present disclosure provides for a device for storing and preferably for holding one or more medical instruments, in particular surgical instruments, and/or implants in an intended arrangement on a perforated instrument holder.

### BRIEF DESCRIPTION OF THE DRAWING FIGURES

Further features, details and advantages of the present disclosure can be found in the drawings and the following description of a preferred embodiment.

FIG. 1 is a perspective view of a perforated instrument holder according to the present disclosure having a perforated holder support structure and a device comprising a base part and an instrument bearing part that can be releasably fastened thereto for storing an instrument (not shown);

FIG. 2 is a perspective view of the base part of the device shown in FIG. 1;

FIGS. 3a-3c are various views illustrating the assembly of the retaining part on the perforated holder support structure of the perforated instrument holder;

FIGS. 4a-4c are side views as well as views from above and below of the perforated instrument holder with the base part assembled;

FIG. 5 shows the process of sliding the instrument bearing part onto the base part already assembled on the perforated holder support structure of the perforated instrument holder; and

FIGS. 6a and 6b show the handling of a release instrument for releasing the base part from the perforated holder support structure of the perforated instrument holder.

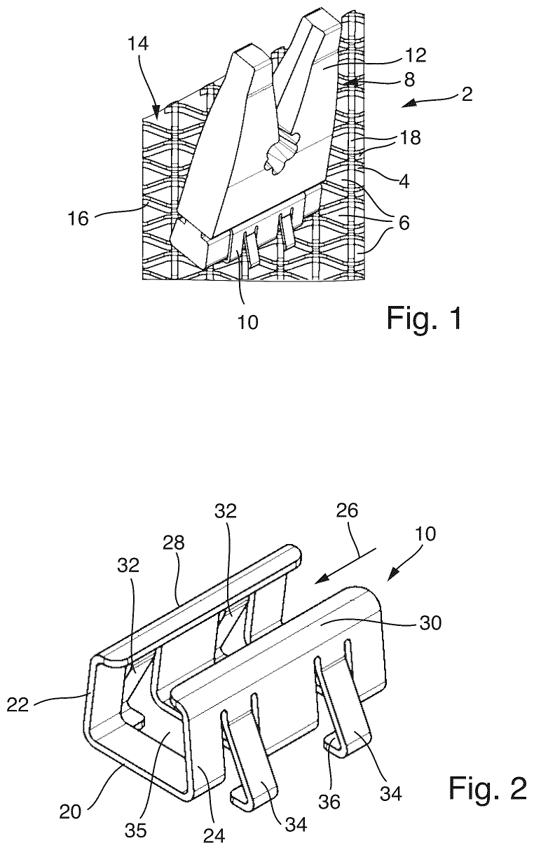

### DETAILED DESCRIPTION

The drawings show an embodiment of a perforated instrument holder 2 according to the present disclosure for medical, in particular surgical, instruments, for use in supplying sterile goods in the medical field. The perforated instrument holder 2 comprises a perforated holder support structure 4 having a plurality of perforated holder openings 6 and a device 8 for storing and preferably for holding an instrument (not shown in the drawings) during the sterile goods cycle. The device 8 comprises a base part 10 and an instrument bearing part 12 that can be releasably fastened thereto. The base part 10 can be releasably fastened to the perforated holder support structure 4 in a manner to be described in more detail below. The perforated holder support structure 4 has a top side 14 facing the device 8 and the instruments (not shown) and a lower side 16 facing away therefrom.

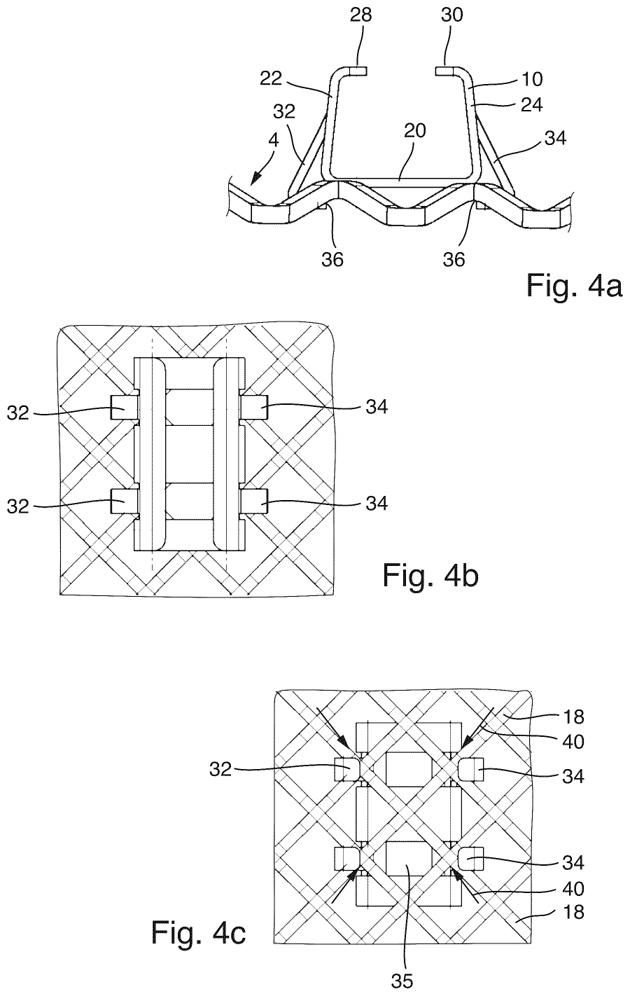

In the example case presented here, the perforated holder support structure 4 is corrugated and thus has a three-dimensional wave structure. The perforated holder support structure 4 is formed, for example, from two groups each consisting of a plurality of parallel connecting pieces 18, wherein the connecting pieces 18 of one group extend at right angles to the connecting pieces 18 of the other group such that they delimit the perforated holder openings 6. The perforated holder support structure 4 consisting of the connecting pieces 18 was initially flat, with the corrugated structure subsequently being developed. Despite the three-dimensional structure, the perforated holder openings 6 can, for example, be described in the present case as being substantially square (cf. FIG. 4b, c), i.e., they have a square shape when projected vertically onto the base surface of the perforated holder support structure 4.

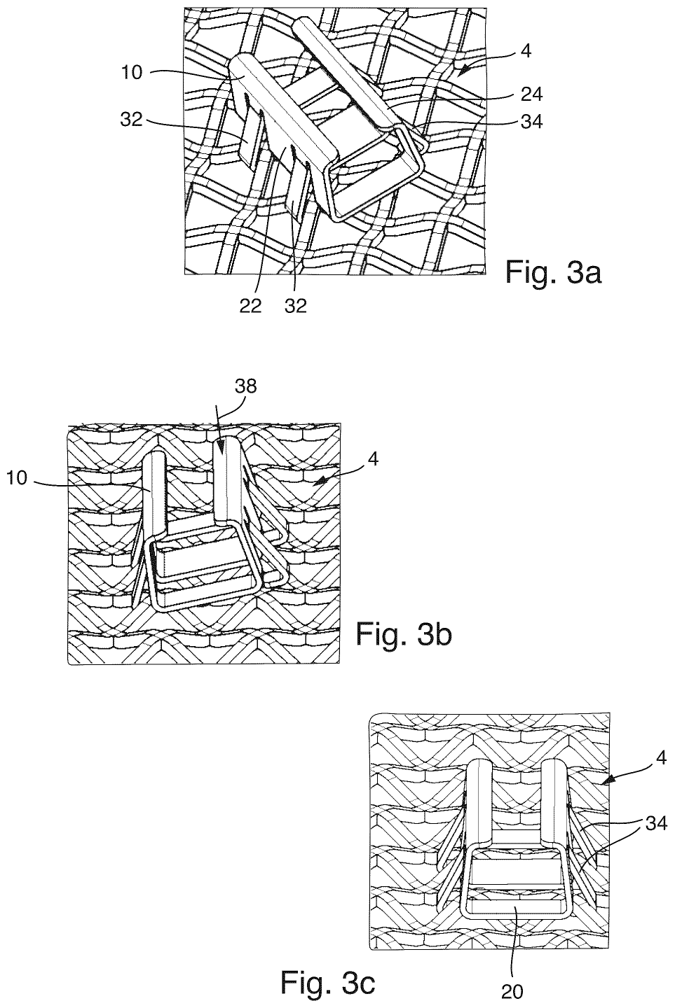

FIG. 2, as well as the other figures, shows the shape and design of the base part 10. In the example case shown here, it was formed from a flat metal sheet material which was brought into the shape shown in FIG. 2 by cutting, punching or bending processes. The base part 10 comprises a main limb 20 which can be placed against the top side 14 of the perforated holder support structure 4 and two opposite side limbs 22, 24 laterally adjoining said main limb, which, in the example case shown, approximately delimit a U shape which is slightly narrowed toward the top when viewed in the direction of an arrow 26. Furthermore, the side limbs 22, 24 have an inwardly bent edge region 28 and 30, respectively, at their top free end. It can also be seen that, proceeding from each of the side limbs 22, 24, two flexible tongues 32, 34 are formed on each side. These flexible tongues 32, 34 are notched out of the material of the former flat material portion by a punch cut and therefore leave a recess 35 in the main limb 20 and in the side limbs 32, 34 of the base part 10. They are bent outward such that they extend obliquely downward at an acute angle in the direction of the perforated holder support structure 4 from a top region of the side limbs 22, 24 and can be resiliently deflected relative to the side limbs 22, 24. Each flexible tongue 32, 34 is bent inward at its free end and forms a rear-engaging region 36 with which a relevant flexible tongue 32, 34 can engage in a perforated holder opening 6 and form a form-fitting retaining connection together with edge regions delimiting the perforated holder opening 6, i.e., with the connecting pieces 18 of the perforated holder support structure 4. This is explained with reference to FIGS. 3a to 3c: FIG. 3a shows the example of the three-dimensionally corrugated perforated holder support structure 4 and the base part 10 with its flexible tongues 32, 34. It can be seen in FIG. 3a that the base part 10 is slightly inclined with respect to a plane of the perforated holder support structure 4 such that the flexible tongues 32 of the side limb 22 shown on the left in FIG. 3a engage in two adjacent perforated holder openings 6 in the region of a wave crest of the perforated holder support structure 4 and hook with the connecting pieces 18 by means of each of their rear-engaging region 36. Proceeding from this assembly situation, only a compressive force in the direction of an arrow 38 shown in FIG. 3b now needs to be exerted on the base part 10 or the other side limb 24 in the direction of the perforated holder support structure 4 such that the opposite flexible tongues 34 run against the connecting pieces 18 with their rear-engaging end regions 36 and are thereby deflected outward and finally reach the rear-engagement situation shown in FIG. 3c, in which the flexible tongues 34 spring back inward after engaging in two perforated holder openings and thereby assume a captive assembly position on the perforated holder support structure 4. In this assembly position, the base part 10 to some extent rests with its main limb 20 in the region of raised portions of the perforated holder support structure 4 and is again arranged in parallel with the plane of the perforated holder support structure 4. However, in the example embodiment shown, the base part does not rest right at the top of the high points of the raised portions of the perforated holder support structure 4, but rather relatively high up on the connecting pieces leading thereto. The high points or apexes of the raised portions penetrate into recesses 35 in the base part, which are created here by the flexible tongues 32 being notched out. If the base part were longer and extended over a plurality of raised portions of the perforated holder support structure, a plurality of such recesses could be formed in the base part to allow the high points or apexes of the raised portions to penetrate or engage in this way.

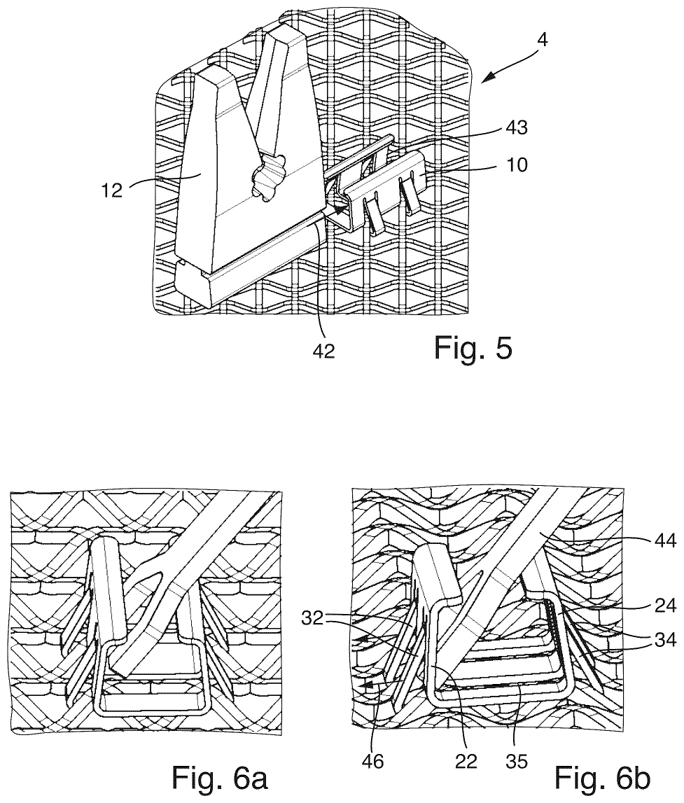

If, in an embodiment that is not shown, the flexible tongues are not adapted to two perforated holder openings of the perforated basket that are arranged directly next to one another, but are designed to engage in perforated holder openings that are further apart from one another, the base part can be provided with one or more additional recesses into which the high points or apexes of the intermediate raised portions of a 3-dimensionally structured perforated body structure can penetrate. This penetration reduces the installation height of the base part above the perforated holder support structure and stabilizes its position with respect to lateral displacement (i.e., in the direction of the arrow in FIG. 5), since the flexible tongues also have a certain degree of resilience in the transverse direction, which is not desired in this direction per se.

Ideally, the surface of the apexes of the raised portions in a corrugated perforated holder support structure and the surface of the main limb of the base part lie in one plane; this allows for a maximum reduction in the installation height of the base part and still allows the instrument bearing part to be inserted without it striking the apexes of the raised portions. For the stability and assembly safety of the instrument bearing part, it is advantageous in particular if its bottom side rests on or abuts the top side of the main limb of the base part.

FIGS. 4a to 4c are further views of the base part 10 assembled on the perforated holder support structure 4.

In FIG. 4c, arrows 40 illustrate how the flexible tongues 32, 34, after penetrating into the perforated holder openings of the three-dimensionally corrugated perforated holder support structure 4 as shown in FIGS. 3a and b, are guided in a self-centering manner along the connecting pieces 18 into the stable assembly position shown due to their spring-back force. The free ends of the flexible tongues 32, 34 are preferably rounded or chamfered, since this reduces the necessary deflection of the flexible tongues 32, 34 when sliding on the connecting pieces of the corrugated perforated holder support structure which extend obliquely thereto. This makes it possible to work with stiffer or stronger spring legs and reduces the risk of damage to the user's gloves or hands during assembly, without having a significant impact on the stability with which the base part is anchored to the perforated holder support structure.

FIG. 5 shows the completed retaining device 8, whereby the instrument bearing part 12 thereof can be pushed onto the base part 10 in the direction of the arrow 42 in parallel with the plane of the perforated holder support structure 4. The base part 10 forms, by means of its top inwardly bent edge regions 28, 30, a dovetail-like sliding seat receptacle for the complementary instrument bearing part 12. However, it should be expressly mentioned that the instrument bearing part 12 and the base part 10 can also be assembled together in another way and releasably fastened to one another. In particular, it is also conceivable to secure the base part 10 together with the pre-assembled instrument bearing part 12 against the perforated holder support structure 4 in the manner described.

However, in order to release the retaining device 8 or the base part 10 from the perforated holder support structure 4, in the example case shown, it is expedient to release the instrument bearing part 12 from the base part 10 that is still assembled so that the flexible tongues 32, 34 can be accessed by means of a release instrument 44 shown in FIGS. 6a and b in order to deflect the flexible tongues 32, 34 outward in the direction of the arrows 46 indicated in FIG. 6b such that they are released from the situation in which they engage behind the connecting pieces 18 of the perforated holder support structure 4 and the base part 10 can initially be pivoted upward away from the perforated holder support structure 4 and then moved to the other side counter to the arrows 46 so that the flexible tongues 34 on the opposite side limb 24 are also released from their rear-engagement situation and the base part 10 can finally be completely removed. Alternatively, with a corresponding design of the length and pre-tension of the flexible tongues 32, 34, it would, however, also be conceivable in principle that the base part 10 is moved without the use of tools—with or without the instrument bearing part 12—to the left in the direction of the arrows 46 so that the flexible tongues 32 shown on the left in FIG. 6b are released from their position in which they engage behind the connecting pieces 18 and the base part can then be pivoted upward and removed from the perforated holder support structure 4 to the opposite side without the use of tools.

1. A instrument holding system for medical instruments, the instrument holding system comprising:
A) a perforated support structure comprising:
i.) a plurality of connecting elements comprising a first group of connecting elements and a second group of connecting elements intersecting the first group of connecting elements at connection points;
ii.) a plurality of holder openings defined between the first group of connecting elements and the second group of connecting elements;
iv.) a top side oriented to face medical instruments held on the perforated support structure; and
v.) a bottom side oriented to face away from medical instruments held on the perforated support structure; and
B) at least one device positionable on the top side of the perforated support structure at various selectable locations on the top side for holding medical instruments in an intended arrangement, the at least one device comprising:
i.) a base part; and
ii.) an instrument bearing part that is releasably fastenable to the base part,
the base part being releasably securable to the perforated support structure,
the base part comprising a bent sheet metal part having a main limb that is positionable against the top side of the perforated support structure and side limbs laterally adjoining the main limb, the side limbs comprising at least a first side limb and a second side limb,
the base part further comprising at least one first flexible tongue extending from the first side limb and at least one second flexible tongue extending from the second side limb,
the at least one first flexible tongue and the at least one second flexible tongue being resiliently deflectable relative to the side limbs,
the base part being positionable on the perforated support structure in a pre-assembly configuration, in which the at least one first flexible tongue extends through a first perforated opening of the plurality of holder openings and hooks behind at least a first connecting element of the plurality of connecting elements, and the at least one second flexible tongue is positioned on the top side of the perforated support structure, and
the base part being further positionable on the perforated support structure in an assembly configuration, in which the at least one first flexible tongue extends through the first perforated opening of the plurality of holder openings and hooks behind at least the first connecting element of the plurality of connecting elements, and the at least one second flexible tongue extends through a second perforated opening of the plurality of holder openings and hooks behind at least a second connecting element of the plurality of connecting elements,
the base part being resiliently deflectable from the pre-assembly configuration to the assembly configuration in response to a compressive force applied on the base part toward the top side of the perforated support structure, such that the base part is manually securable to the perforated support structure in the assembly configuration without a tool.

2. The instrument holding system according to claim 1, wherein the at least one first flexible tongue and the at least one second flexible tongue are notched out of the bent sheet metal part.

3. The instrument holding system according to claim 2, wherein the at least one first flexible tongue is notched out of at least the first side limb, and the at least one second flexible tongue is notched out of at least the second side limb.

4. The instrument holding system according to claim 3, wherein the at least one first flexible tongue is notched out of the first side limb and the main limb, and the at least one second flexible tongue is notched out of the second side limb and the main limb.

5. The instrument holding system according to claim 1, wherein the at least one first flexible tongue and the at least one second flexible tongue are resiliently deflectable relative to the side limbs in such a way that by exerting the compressive force on the base part toward the top side of the perforated support structure, the at least one second flexible tongue slides against the top side and deflects outward until the at least one second flexible tongue aligns with the second perforated opening of the plurality of holder openings, at which point the at least one second flexible tongue penetrates through the perforated support structure and hooks behind at least the second connecting element of the plurality of connecting elements.

6. The instrument holding system according to claim 1, wherein each of the at least one first flexible tongue and the at least one second flexible tongue comprises a free end that forms a rear-engaging region for engaging the bottom side of the perforated support structure.

7. The instrument holding system according to claim 6, wherein each of the at least one first flexible tongue and the at least one second flexible tongue further comprises a longitudinal region that forms an angle of at least 90° with the rear-engaging region.

8. The instrument holding system according to claim 6, wherein each free end forms a run-on slope, which, when the base part is pressed toward the top side of the perforated support structure, runs against an edge region delimiting one of the plurality of holder openings and is deflected in order to latch with the edge region into a rear-engagement position.

9. The instrument holding system according to claim 1, wherein the main limb, the first side limb, and the second side limb form a U shape.

10. The instrument holding system according to claim 9, wherein the base part forms a receptacle for the instrument bearing part, the receptacle being delimited by the main limb, the first side limb and the second side limb.

11. The instrument holding system according to claim 1, wherein the instrument bearing part is a polymeric plastics part.

12. The instrument holding system according to claim 1, wherein the instrument bearing part is dimensionally stable but has resiliently deflectable retaining regions for the medical instruments.

13. The instrument holding system according to claim 1, wherein:
each of the plurality of holder openings is rectangular with a maximum side length, and
each of the plurality of connecting elements has a thickness that is less than the maximum side length.

14. The instrument holding system according to claim 1, wherein:
the instrument holding system defines a longitudinal direction and a transverse direction, and
each of the plurality of connecting elements is oriented at an angle of 45° relative to the longitudinal direction and to the transverse direction.

15. The instrument holding system according to claim 1, wherein:
the perforated support structure comprises a 3-dimensional structure with high points or peaks oriented to face medical instruments held on the perforated support structure, and
the base part is fastenable to the perforated support structure at the high points or peaks.

16. The instrument holding system according to claim 1, wherein:
the at least one first flexible tongue extending from the first side limb comprises two first flexible tongues separated by a first distance,
the at least one second flexible tongue extending from the second side limb comprises two second flexible tongues separated by a second distance,
the connection points are separated by from one another by a connection point distance, and
each of the first distance and the second distance is an integer multiple of the connection point distance.

17. The instrument holding system according to claim 1, further comprising a release instrument that is insertable into the base part and configured to deflect the at least one first flexible tongue or the at least one second flexible tongue to release the base part from the assembly configuration.

18. The instrument holding system according to claim 1, wherein the perforated support structure comprises a perforated instrument basket, an insert for a perforated instrument basket, or an insert for an instrument container.

19. A device for holding one or more medical instruments in an intended arrangement on an instrument holding system, the device comprising:
a base part; and
an instrument bearing part that is releasably fastenable to the base part,
the base part comprising a bent sheet metal part having a main limb and side limbs laterally adjoining the main limb, the side limbs comprising at least a first side limb and a second side limb,
the base part further comprising at least one first flexible tongue extending from the first side limb and at least one second flexible tongue extending from the second side limb,
the at least one first flexible tongue and the at least one second flexible tongue being resiliently deflectable relative to the side limbs,
the at least one first flexible tongue comprising a first free end forming a first hook,
the at least one second flexible tongue comprising a second free end forming a second hook, and
the first hook and the second hook extending inwardly toward one another.

20. The device for holding one or more medical instruments according to claim 19, wherein the at least one first flexible tongue is notched out of at least the first side limb, and the at least one second flexible tongue is notched out of at least the second side limb.

21. The device for holding one or more medical instruments according to claim 20, wherein the at least one first flexible tongue is notched out of the first side limb and the main limb, and the at least one second flexible tongue is notched out of the second side limb and the main limb.

22. The device for holding one or more medical instruments according to claim 21, wherein the base part defines a recess extending between the at least one first flexible tongue and the at least one second flexible tongue, the recess being defined where the at least one first flexible tongue is notched out of the main limb, and where the at least one second flexible tongue is notched out of the main limb.
